(19) United States
(12) Patent Application Publication
(10) Pub. No.: US 2004/0170287 A1
(43) Pub. Date: Sep. 2, 2004

(54) Accoustic wave amplifier/attenuator apparatus, pipe system having the same and manufacturing method of the pipe system

(76) Inventors: Tetsushi Biwa, Nagoya-city (JP); Taichi Yazaki, Okazaki-city (JP); Yusuke Tashiro, Nagoya-city (JP); Yuki Ueda, Nagoya-city (JP); Motoki Kozuka, Nagoya-city (JP)

Correspondence Address: POSZ & BETHARDS, PLC, 11250 ROGER BACON DRIVE, SUITE 10, RESTON, VA, 20190, US

(21) Appl. No.: 10/786,121

(22) Filed: Feb. 26, 2004

(30) Foreign Application Priority Data
Feb. 27, 2003 (JP) 2003-51756

(51) Int. Cl. A61F 11/06; G10K 11/16; H03B 29/00

(52) U.S. Cl. 381/71.5; 381/71.7; 181/224

(57) ABSTRACT

A sound source device is connected to one end of a pipe arrangement to supply sound into the pipe arrangement. Regenerators are installed to the pipe arrangement. Each regenerator serves as an energy converter that converts energy of an acoustic wave of the sound, which is propagated in the pipe arrangement, into another form of energy. Acoustic wave amplifier/attenuator apparatuses are provided in the pipe arrangement at a corresponding location between the sound source device and a corresponding one of the regenerators. Each acoustic wave amplifier/attenuator apparatus uses thermoacoustic effect to amplify or attenuate the acoustic wave and includes a cold heat exchanger, a hot heat exchanger and a stack that is held between the cold heat exchanger and the hot heat exchanger.

### CROSS REFERENCE TO RELATED APPLICATION

This application is based on and incorporates herein by reference Japanese Patent Application No. 2003-51756 filed on Feb. 27, 2003.

### BACKGROUND OF THE INVENTION

1. Field of the Invention

The present invention relates to an acoustic wave amplifier/attenuator apparatus, which uses thermoacoustic effect to amplify or attenuate an acoustic intensity of an acoustic wave, which is supplied from an external sound source (e.g., a speaker, a piston, a spontaneous thermoacoustic oscillation generator). The present invention further relates to a pipe system that has such an acoustic wave amplifier/attenuator apparatus and also relates to a manufacturing method of the pipe system.

2. Description of Related Art

Ceperley has proposed a method for amplifying an acoustic intensity of an acoustic wave in “Gain and efficiency of a short traveling-wave heat engine,” 77 J. Acoust. Soc. Am., pp. 1239-1294 (1985), the subject matter of which is incorporated by reference herein. In this method, the acoustic intensity is amplified through a stack, which has a thermal gradient generated by an external heat source in an axial direction of the stack.

A speaker is connected to one end of an elongated pipe. Furthermore, the stack, which has the thermal gradient, is inserted in the pipe at a location downstream of the speaker in a propagating direction of an acoustic wave outputted from the speaker. In such a case, when a traveling acoustic wave is supplied from the speaker and passes through the stack, energy exchange takes place through a Stirling cycle between the acoustic wave and the heat energy applied to the stack from the external heat source. Ceperley has predicted that amplification or attenuation of the acoustic intensity of the acoustic wave takes place through the stack depending on a positive sign or a negative sign of the temperature gradient generated in the stack. This is due to the fact that pressure and velocity oscillations have essentially the same phase in a case of a traveling acoustic wave like those in a case of oscillating fluid in a stack of a Stirling engine. Ceperley has stated that when the acoustic wave passes from the low temperature side to the high temperature side through the stack, an amplification ratio of the acoustic intensity would reach, at most, a temperature ratio between the high temperature side and the low temperature side in an ideal condition like in the case of the previously known Stirling engine. However, Ceperley has failed experiments to prove this point, so that Ceperley has failed to prove the high amplification of the acoustic wave beyond the temperature ratio by the heat. This failure is due to the following fact. That is, isothermal heat exchange needs to occur in the stack to form the Stirling cycle. However, when each flow channel in the stack is narrowed to achieve the isothermal heat exchange, an energy loss, which is caused by viscosity of fluid present in the stack, is increased.

Petculescu has proposed positioning of the above stack in a velocity node of a standing wave in a pipe in “Traveling-wave amplification in a variable standing wave ratio device”, Acoustics Research Letters on Line, vol. 3, pp 71-76 (2002), the subject matter of which is incorporated by reference herein.

Furthermore, Japanese Patent No. 3015786, Japanese Unexamined Patent publication No. 2001-521125 (corresponding to U.S. Pat. No. 6,314,740), Japanese Unexamined Patent Publication No. 2002-31423 and Japanese Unexamined Patent Publication No. 2002-535597 (corresponding to U.S. Pat. No. 6,032,464) also disclose relevant technologies.

In the case of Petculescu, similar to Ceperley, energy of the traveling wave is utilized, but energy of the standing wave is not utilized. In order to use the energy of the traveling wave, the isothermal heat exchange needs to be performed in the stack. However, in order to perform the isothermal heat exchange, an energy loss, which is caused by the viscosity of the fluid in the stack, cannot be ignored. When a position of the stack is deviated from a velocity node, the amplification ratio is disadvantageously substantially decreased.

### SUMMARY OF THE INVENTION

The present invention addresses the above disadvantages. Thus, it is an objective of the present invention to provide a pipe system, which amplifies or attenuates an acoustic intensity of an acoustic wave and addresses the above disadvantages. It is another objective of the present invention to provide a manufacturing method of such a pipe system. It is a further objective of the present invention to provide an acoustic wave amplifier/attenuator apparatus, which amplifies or attenuates an acoustic intensity of an acoustic wave and addresses the above disadvantages.

To achieve the objectives of the present invention, there is provided a pipe system that includes a pipe arrangement, a sound source, at least one energy converter and at least one acoustic wave amplifier/attenuator apparatus. The sound source device is connected to the pipe arrangement to supply sound into the pipe arrangement. Each energy converter converts energy of an acoustic wave of the sound, which is propagated in the pipe arrangement, into another form of energy. Each acoustic wave amplifier/attenuator apparatus is provided in the pipe arrangement at a corresponding location between the sound source device and a corresponding one of the at least one energy converter and uses thermoacoustic effect to amplify or attenuate the acoustic wave. Furthermore, each acoustic wave amplifier/attenuator apparatus includes a cold heat exchanger, a hot heat exchanger and a stack. The stack is held between the cold heat exchanger and the hot heat exchanger.

To achieve the objectives of the present invention, there is also provided a manufacturing method of a pipe system. In the method, a sound source device is installed to a pipe arrangement. Then, at least one energy converter is installed to the pipe arrangement. Each energy converter converts energy of an acoustic wave, which is propagated in the pipe arrangement, into another form of energy. Thereafter, at least one acoustic wave amplifier/attenuator apparatus is installed in the pipe arrangement at a corresponding location between the sound source device and a corresponding one of the at least one energy converter. Each acoustic wave amplifier/attenuator apparatus uses thermoacoustic effect to amplify or attenuate the acoustic wave. Furthermore, each acoustic wave amplifier/attenuator apparatus includes a cold heat exchanger, a hot heat exchanger and a stack. The stack is held between the cold heat exchanger and the hot heat exchanger.

### BRIEF DESCRIPTION OF THE DRAWINGS

The invention, together with additional objectives, features and advantages thereof, will be best understood from the following description, the appended claims and the accompanying drawings in which:

FIG. 1 is a schematic view showing a structure of a pipe system according to an embodiment of the present invention;

FIG. 2A is a partial schematic perspective view of a modification of a stack of an acoustic wave amplifier/attenuator apparatus of the pipe system of FIG. 1;

FIG. 2B is a partial enlarged end view seen in a direction IIB in FIG. 2A;

FIG. 3 is a schematic view showing one experimental case of the embodiment;

FIG. 4 is a graph showing a relationship between a phase difference and an axial position in a pipe arrangement;

FIG. 5 is a graph showing a relationship between an acoustic intensity and an axial position in a pipe arrangement for various cases;

FIG. 6 is a graph showing a relationship between an amplification ratio and a phase difference at a center of the stack in a state where a parameter on is about 4.9; and

FIG. 7 is a graph showing a relationship between an amplification ratio and a phase difference at the center of the stack in a state where a parameter on is about 0.18.

### DETAILED DESCRIPTION OF THE INVENTION

An embodiment of the present invention will be described with reference to the accompanying drawings.

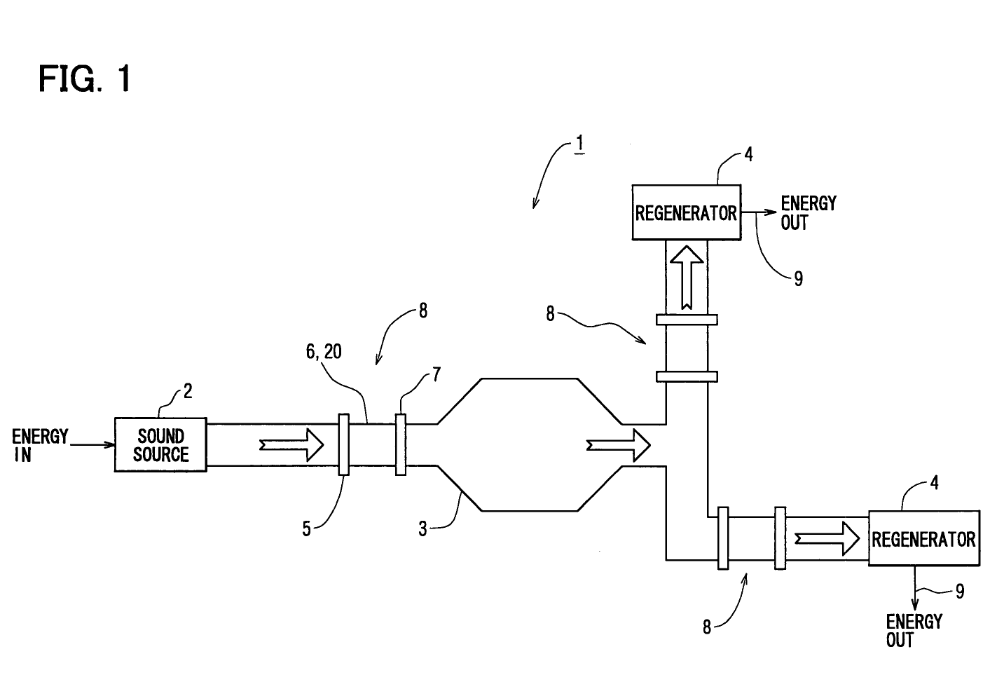

FIG. 1 is a schematic view of a pipe system 1 of the present embodiment. A working gas of about 0.1-3 MPa is filled in a pipe arrangement 3 of the pipe system 1. Helium gas, nitrogen gas, air, hydrogen gas or any other gas mixture can be used as the working gas. A sound source device (e.g., a speaker, a piston, a spontaneous thermoacoustic oscillation generator that makes spontaneous thermoacoustic oscillation) 2 is connected to one end of the pipe arrangement 3. The pipe arrangement 3 can be generally made from one or more pipes, each of which has a circular cross section and is made of, for example, stainless steel. An inner diameter of the pipe arrangement 3 is not necessarily constant. Furthermore, the pipe arrangement 3 can have a branch or can have a curved shape.

Multiple (two in this embodiment) regenerators 4 are provided in the pipe system 1. Each regenerator 4, which serves as an energy converter of the present invention and converts energy of an acoustic wave into heat energy, is connected to a corresponding end of the pipe arrangement 3. An interface 9 is connected to the corresponding regenerator 4 to externally output heat (cold heat or cold energy) of the regenerator 4. The interface 9 can be, for example, a circulation device for circulating a heat medium.

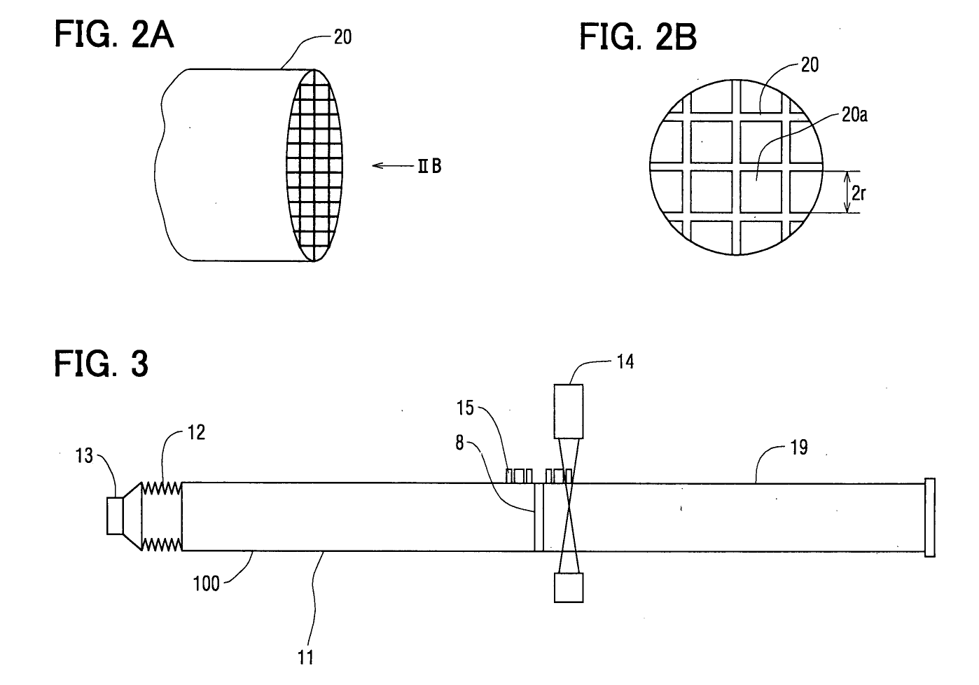

Multiple (three in this embodiment) acoustic wave amplifier/attenuator apparatuses 8 are provided in the pipe arrangement 3. In the pipe arrangement 3, each acoustic wave amplifier/attenuator apparatus 8 includes a stack 6, which is interposed between a clod heat exchanger 5 and a hot heat exchanger 7. The stack 6 includes a stack of a plurality of screen meshes (also referred to as meshed thin metal plates), which are stacked in, for example, an axial direction of the pipe arrangement 3 and form a plurality of flow channel or a plurality of flow passages. Alternative to the stack 6, a stack 20 of FIGS. 2A and 2B may be provided. The stack 20 has a honeycomb structure and is made from a ceramic material or a web-like thin metal plate material. Thus, the stack 20 has a plurality of parallel flow channels 20a. In FIGS. 2A and 2B, although a cross-sectional shape of each flow channel 20a is square, the cross-sectional shape of each flow channel 20a can have any other appropriate shape, such as a circular shape, an oblong shape, a polygonal shape (including any rectangular shape or any other polygonal shape). The hot heat exchanger 7 may include a plurality of thin metal plates, which are arranged at small intervals. Alternatively, the hot heat exchanger 7 may include a stack of a plurality of screen meshes. The hot heat exchanger 7 is heated by, for example, a heater, flames or waste heat. The clod heat exchanger 5 has a structure similar to that of the hot heat exchanger 7 and is cooled by, for example, room temperature coolant or cooling water (not shown). When a wavelength of the acoustic wave conducted in the pipe arrangement 3 is set to 1, an axial center of the stack 6, 20 should be arranged at a position that is located within ±0.2 from a corresponding velocity node formed in the pipe arrangement 3.

The acoustic wave amplifier/attenuator apparatus 8 is not substantially directly connected to external devices except the heat exchangers, which supply the heats to the hot heat exchanger and the cold heat exchanger. Although the acoustic wave amplifier/attenuator apparatus 8 can convert energy of the acoustic wave into, for example, heat energy, the acoustic wave amplifier/attenuator apparatus 8 is used to amplify or to attenuate the acoustic wave present in the pipe arrangement 3 in this embodiment. Thus, when the acoustic wave amplifier/attenuator apparatus 8 is used to convert the energy of the acoustic wave into, for example, the heat energy, it will result in a reduction in an amplifying efficiency or an attenuating efficiency of the acoustic wave amplifier/attenuator apparatus 8. Thus, an interface, such as the interface 9 used to output the energy from the regenerator 4, is not connected to the acoustic wave amplifier/attenuator apparatus 8.

In general, when a sound source is provided on an upstream side of the acoustic wave amplifier/attenuator apparatus, at which side the clod heat exchanger is located, in a propagating direction of the acoustic wave, the acoustic wave amplifier/attenuator apparatus acts as an amplifier. In contrast, when a sound source is provided on the upstream side of the acoustic wave amplifier/attenuator apparatus, at which side the hot heat exchanger is located, in the propagating direction of the acoustic wave, the acoustic wave amplifier/attenuator apparatus acts as an attenuator.

An exemplary experimental case of the present embodiment will be described with reference to FIG. 3. A pipe arrangement 100 includes stainless pipes 11, 19, each of which has an inner diameter of 24 mm and a wall thickness of 0.5 mm. A speaker 13, which serves as the sound source device 2 of FIG. 1, is connected to one end of the upstream side pipe 11 through a metal bellows 12. Although the stainless pipes 11, 19 are used in a normal situation, glass pipes, each of which has an inner diameter of 21 mm and a wall thickness of 2 mm, are used as the pipes 11, 19 to allow measurement of a flow velocity of the working gas in the pipe arrangement through a laser Doppler flowmeter (having a photomultiplier) 14. An entire length of the pipe arrangement 100 is 3.3 m, and the atmospheric pressure air is filled in the pipe arrangement 100 as the working gas. A vibration frequency of the speaker 13 is 103 Hz, which is a frequency that achieves one full wavelength resonance in the pipe arrangement 100. Because of the above settings, the acoustic wave amplifier/attenuator apparatus 8 is arranged generally in the center of the pipe arrangement 100. However, a relative axial position of the acoustic wave amplifier/attenuator apparatus 8 in the pipe arrangement 100 can be shifted from one position to another position without changing the entire length of the pipe arrangement 100 by appropriately changing both the pipes 11, 19, which are arranged on the upstream side and the downstream side, respectively, of the acoustic wave amplifier/attenuator 8. The stack of the acoustic wave amplifier/attenuator 8, which corresponds to the stack 6 of FIG. 1, includes a plurality of stainless steel screen meshes (mesh number 60), which are stacked one after the other for 2 cm.

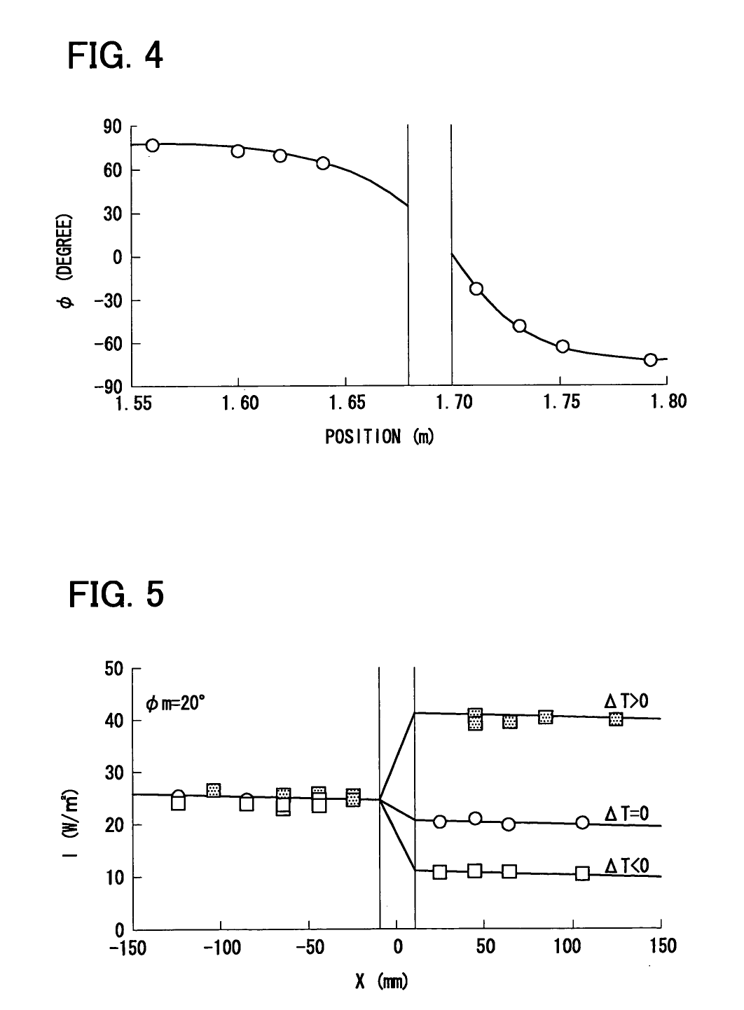

FIG. 4 shows an experimental result that indicates an axial distribution of a phase difference (phase angle) between the pressure and the flow velocity, which are measured by a pressure sensor 15 and the laser Doppler flowmeter 14, respectively. The experimental result is obtained by positioning the center of the stack at a location that is spaced 1.69 m from a vibrating surface of the speaker 13 while maintaining the entire stack at the room temperature. In the pipe arrangement 100, an axial position (1.70 m away from the vibrating surface of the speaker 13), at which the phase difference is zero, is a velocity node. Thus, it should be noted that the axial center of the stack is positioned slightly ahead of the velocity node on the upstream side of the velocity node. In this instance, the phase difference at the axial center of the stack of the acoustic wave amplifier/attenuator 8 is about +20 degrees. Since the phase difference progressively changes in the axial direction in the pipe arrangement 100, as shown in FIG. 4, the phase difference at the center of the stack of the acoustic wave amplifier/attenuator 8 can be changed to any other desired value by changing the axial position of the stack. However, it is not necessary to change the axial position of the stack to change the phase difference at the center of the stack. More specifically, the phase difference at the center of the stack can be alternatively changed by increasing or decreasing the vibration frequency of the sound source device.

FIG. 5 shows various acoustic intensities (denoted by “I” in FIG. 5) of the acoustic waves, which are measured while the phase difference at the center of the stack is kept at +20 degrees. More specifically, FIG. 5 shows the acoustic intensity measured under no temperature gradient (ΔT=0), the acoustic intensity measured under the positive temperature gradient (ΔT>0) where the acoustic wave is propagated from the low temperature side to the high temperature side, and the acoustic intensity measured under the negative temperature gradient (ΔT<0). Here, when the temperature gradient exists (ΔT>0 or ΔT<0), one axial end of the stack is cooled at the room temperature, and the other axial end of the stack is heated at 290 degrees Celsius. Furthermore, the acoustic intensity of the acoustic wave, which is inputted to the stack, is kept constant. As shown in FIG. 5, when the temperature gradient is positive (ΔT>0), the acoustic intensity of the acoustic wave is amplified, i.e., is increased. By contrast, when the temperature gradient is negative (ΔT<0), the acoustic intensity of the acoustic wave is attenuated, i.e., is decreased. Thus, the acoustic wave amplifier/attenuator 8 uses the heat energy to amplify or to attenuate the acoustic intensity of the acoustic wave. Hereinafter, an acoustic wave amplification ratio refers to a value, which is obtained by dividing the acoustic intensity of the output acoustic wave, which is outputted from the stack, by the acoustic intensity of the input acoustic wave, which is inputted to the stack (i.e., Intensity output/Intensity input). When the amplification ratio is greater than 1, the acoustic intensity of the acoustic wave is amplified. When the amplification ratio is less than 1, the acoustic intensity of the acoustic wave is attenuated. A non-dimensional parameter ωτ of the stack used in the above experiment is about 0.2, which is relatively small. Here, the parameter an indicates a degree of thermal exchange between the oscillating gas and a solid wall of the stack. Furthermore, “ω” is an angular frequency of the acoustic wave, and “τ” is defined as τ=r²/2α, where “r” is a radius of the corresponding flow channel or flow passage (or one half of a narrowest transverse length 2r of the flow channel 20a measured in a direction perpendicular to the axial direction of the flow channel 20a, as in the case of FIGS. 2A, 2B), and α is a thermal diffusivity of the acoustic medium, i.e., oscillating fluid. When the parameter ωτ is relatively small (e.g., the above case of ωτ=0.2), only the energy of the traveling wave is available for the amplification/attenuation. As a result, the maximum amplification ratio is limited to the temperature ratio (=560° K./300° K.) and cannot exceed the temperature ratio in such a case.

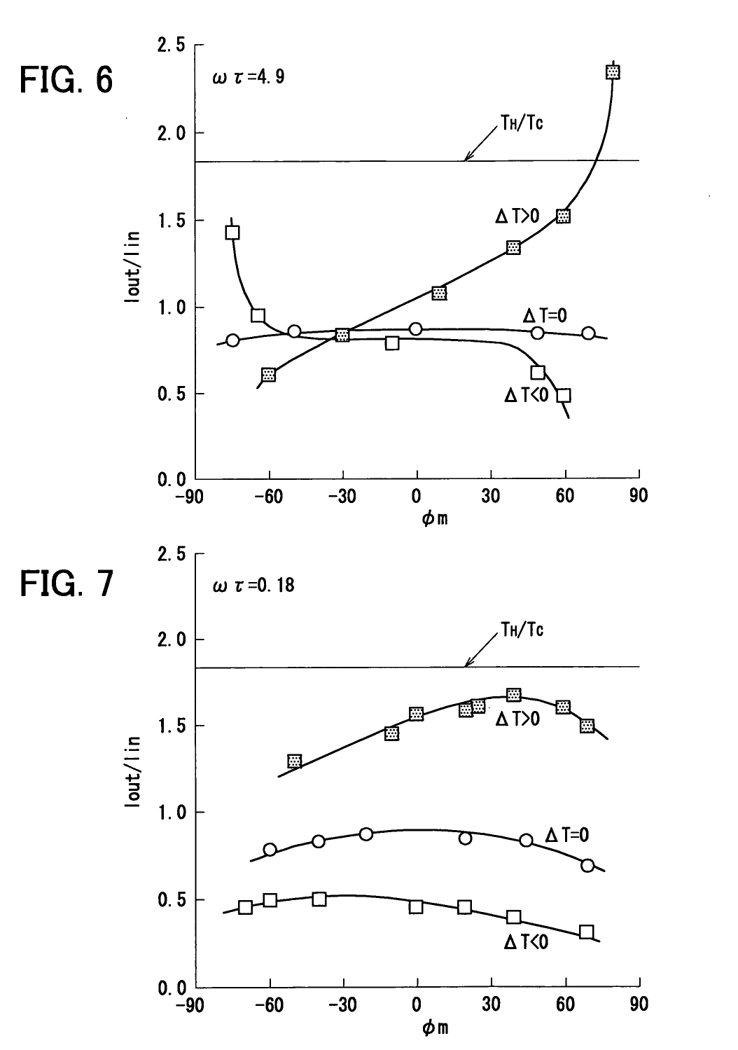

Next, another exemplary experimental case, in which a ceramic honeycomb body (also referred as a honeycomb ceramics) is used to form the stack similar to the stack 20 of FIGS. 2A and 2B, will be described. The honeycomb body has a pore density of 200 pores per square centimeter. A radius of each flow channel (or one half of a narrowest transverse length of the flow channel) of the stack made of the honeycomb body is greater than that of the stack made of the stainless steel screen meshes. An experiment similar to that of the above case is performed, and the result of this experiment is shown in FIG. 6. In FIG. 6, an axis of ordinates indicates the amplification ratio (Intensity output/Intensity input), and an axis of abscissas indicates the phase difference at the center of the stack. The parameter ωτ (indicating the degree of thermal exchange between the oscillating gas and the solid wall of the stack) of the stack used in this experiment is about 4.9, so that both the traveling wave and standing wave can contribute to the above-described energy conversion. When the stack is placed in the velocity node, only the traveling wave can contribute to the energy conversion. However, even in such a situation, amplification/attenuation of the acoustic intensity of the acoustic wave can be monitored based on the positive/negative sign of the temperature gradient. In a case where the stack is displaced from the velocity node to utilize the standing wave, when the temperature gradient is positive (ΔT>0), and the phase difference is positive, the amplification ratio is increased and can even exceed the temperature ratio (TH/TC) as is clearly shown in FIG. 6. This is due to the contribution of the energy conversion of the standing wave. By contrast, when the temperature gradient is positive (ΔT>0), but the phase difference is negative, the acoustic wave is attenuated, as shown in FIG. 6. This is due to the fact that the amplification/attenuation of the acoustic intensity of the acoustic wave depends on a combination of the sign (i.e., positive sign or negative sign) of the temperature gradient and the sign (i.e., positive sign or negative sign) of the phase difference in the case of the standing wave energy conversion unlike the traveling wave energy conversion. By using this fact, the amplification/attenuation of the acoustic intensity of the acoustic wave can be changed even in a case where it is difficult to change the sign (i.e., positive sign or negative sign) of the temperature gradient.

As described above, according to the present embodiment, the acoustic intensity of the acoustic wave, which is supplied from the external sound source, such as the spontaneous thermoacoustic oscillation generator, the speaker or the piston, can be amplified to provide a large acoustic wave output or can be alternatively attenuated. In this way, even when electric power, drive power or a high temperature heat source is not present to drive the sound source, the required acoustic wave output can be provided through the thermoacoustic effect without requiring any movable component. Furthermore, through a combination of a plurality of acoustic wave amplifiers (i.e., the acoustic wave amplifier/attenuator apparatuses used as the acoustic wave amplifiers), a relatively large acoustic wave output, which cannot be made by a single acoustic wave amplifier (i.e., the acoustic wave amplifier/attenuator apparatus used as the acoustic wave amplifier), can be provided. Even in a case of the sound source, which has a movable component, only a smaller amount of electric power or a smaller amount of drive power is required to provide the same acoustic wave output in comparison to the sound source, which does not use the thermoacoustic effect. This results in a reduction in the required energy and contributes to a longer lifetime. In a case of the spontaneous thermoacoustic oscillation generator, a relatively large acoustic wave output, which cannot be achieved by a single stack, can be made at a lower temperature. Furthermore, a relatively large acoustic output can be actively attenuated by heat through the acoustic wave amplifier/attenuator apparatus of the present embodiment without fully relying on acoustical material or interference effect, which attenuates the acoustic wave. Furthermore, in the acoustic wave amplifier/attenuator apparatus, which uses the thermoacoustic effect, there is no movable component involved, so that the acoustic wave amplifier/attenuator apparatus can advantageously achieve a reduction in manufacturing costs, an increase in the lifetime of the apparatus, and an improved reliability of the apparatus.

In the above embodiment, as long as the pipe arrangement can enclose the acoustic medium (gas, such as air), a material, a cross-sectional shape, an inner diameter, a configuration and presence of a branch of the pipe arrangement are not limited to the above describe ones and thus can be changed to any desired ones.

The sound source device (or simply referred to as the sound source) can be any appropriate one, such as the spontaneous thermoacoustic oscillation generator, the speaker or the piston, as long as it can convert corresponding energy into the acoustic wave energy. Furthermore, an arrangement, which introduces an externally generated acoustic wave into the pipe arrangement, is also considered as the sound source device.

The acoustic wave introduced from the sound source device into the pipe arrangement forms the standing wave and the traveling wave in the pipe arrangement.

The energy converter, which converts energy of the acoustic wave into another form of energy, can be the regenerator (converting the acoustic wave into the heat energy) like a heat pump. Also, the energy converter can be a microphone (converting the acoustic wave into electric energy) or a piston (converting the acoustic wave into mechanical energy).

As described above, the acoustic wave amplifier/attenuator apparatus of the above embodiment can amplify the acoustic wave propagated in the pipe arrangement beyond the temperature ratio (Th/Tc) between the hot heat exchanger and the cold heat exchanger. This is due to the fact that both the traveling wave and standing wave propagated in the pipe arrangement are used to amplify the acoustic wave. In order to use both the traveling wave and standing wave in the amplification of the acoustic wave, the parameter ωτ of the stack is desirably set in a range of 1-20. More desirably, the parameter ωτ of the stack is set in a range of 2-10. Further desirably, the parameter ωτ of the stack is set in a range of 3-7. As described above, when the parameter ωτ is less than 1, the acoustic wave present in the pipe arrangement cannot be amplified beyond the temperature ratio (Th/Tc), as shown in FIG. 7.

Furthermore, as described above, in order to utilize both the traveling wave and the standing wave in the energy conversion, the axial center of the stack is desirably displaced from the node of the standing wave (i.e., the location where the phase difference Φ between the pressure and the flow velocity is zero). This is due to the fact that the standing wave cannot be used at the node although the use of the traveling wave can be maximized at the node.

It is understood that the acoustic wave can be amplified only by the traveling wave even when the center of the stack is placed in the location where the phase difference is zero.

At the time of amplifying the acoustic wave, it is desirable that the phase difference Φ is positive, and the temperature gradient is provided in the stack from the low temperature side to the high temperature side in the propagating direction of the acoustic wave. This is due to the fact that both the traveling wave and the standing wave can contribute to the amplification of the acoustic wave with this arrangement. At the time of attenuating the acoustic wave, it is desirable that the phase difference Φ is negative, and the temperature gradient is provided in the stack from the high temperature side to the low temperature side in the propagating direction of the acoustic wave. This is due to the fact that both the traveling wave and the standing wave can contribute to the attenuation of the acoustic wave with this arrangement.

The following table 1 shows a relationship between the phase difference Φ and the temperature gradient.

TABLE 1

| STANDING WAVE | | Φ | |
|---|---|---|---|
| ENERGY CONVERSION | | POSITIVE | NEGATIVE |
| TEMPER- | POSITIVE | AMPLIFICATION | ATTENUATION |
| ATURE | NEGATIVE | ATTENUATION | AMPLIFICATION |
| GRADIENT | | | |
| TRAVELING WAVE | | NOT DEPEND ON SIGN OF Φ | |
| ENERGY CONVERSION | | | |
| TEMPER- | POSITIVE | AMPLIFICATION | AMPLIFICATION |
| ATURE | NEGATIVE | ATTENUATION | ATTENUATION |
| GRADIENT | | | |

With reference to the wavelength (λ) of the standing wave, it is desirable that the location of the center of the stack is located within a range of ±0.2λ from the node but is displaced from the node. This setting can limit an increase in loss of energy caused by the viscosity in the stack. It is more desirable that the location of the center of the stack is located within a range of ±0.125λ from the node but is displaced from the node.

When the cold heat exchanger is located on the upstream side of the stack in the propagating direction of the acoustic wave in the pipe arrangement (thereby the hot heat exchanger being located on the downstream side of the stack), the pressure oscillation takes place such that the acoustic wave is amplified in the stack. When multiple acoustic wave amplifiers (i.e., the acoustic wave amplifier/attenuator apparatuses used as the acoustic wave amplifiers), each of which has the above structure, are used, the sufficiently large output can be outputted from the energy converter even when the power of the sound source is relatively small.

In contrast, when the cold heat exchanger is located on the downstream side of the stack in the propagating direction of the acoustic wave in the pipe arrangement (thereby the hot heat exchanger being located on the upstream side of the stack), the pressure oscillation takes place such that the acoustic wave is attenuated in the stack. When multiple acoustic wave attenuators (i.e., the acoustic wave amplifier/attenuator apparatuses used as the acoustic wave attenuators), each of which has the above structure, are used, the acoustic wave of excessively large energy can be adapted to coincide with a capacity of the energy converter.

The acoustic wave amplifier(s) and the acoustic wave attenuator(s) can be provided together in a single pipe arrangement.

Use of the multiple acoustic wave amplifier/attenuator apparatuses is effective when the temperature of the heat supplied to the hot heat exchanger and/or the cold heat exchanger is limited. Particularly, when the temperature of the hot heat exchanger is relatively low, the use of the multiple acoustic wave amplifier/attenuator apparatuses can achieve the relatively large output. However, it should be noted that only one acoustic wave amplifier/attenuator apparatus can be provided in the pipe system, if desired.

Additional advantages and modifications will readily occur to those skilled in the art. The invention in its broader terms is therefore not limited to the specific details, representative apparatus, and illustrative examples shown and described.

What is claimed is:

1. A pipe system comprising:
a pipe arrangement;
a sound source device that is connected to the pipe arrangement to supply sound into the pipe arrangement;
at least one energy converter that converts energy of an acoustic wave of the sound, which is propagated in the pipe arrangement, into another form of energy; and
at least one acoustic wave amplifier/attenuator apparatus that is provided in the pipe arrangement at a corresponding location between the sound source device and a corresponding one of the at least one energy converter and uses thermoacoustic effect to amplify or attenuate the acoustic wave, wherein each acoustic wave amplifier/attenuator apparatus includes:
a cold heat exchanger;
a hot heat exchanger; and
a stack that is held between the cold heat exchanger and the hot heat exchanger.

2. The pipe system according to claim 1, wherein ωτ of the stack of each acoustic wave amplifier/attenuator apparatus is in a range of 1-20.

3. The pipe system according to claim 1, wherein an axial center of the stack of each acoustic wave amplifier/attenuator apparatus is displaced from a node of a standing wave generated in the pipe arrangement.

4. The pipe system-according to claim 1, wherein:
the sound source device is a spontaneous thermoacoustic oscillation generator; and
at least one of the at least one energy converter is a regenerator.

5. The pipe system according to claim 1, wherein each acoustic wave amplifier/attenuator apparatus has an acoustic wave amplification ratio that is greater than a temperature ratio between the hot heat exchanger and the cold heat exchanger of the acoustic wave amplifier/attenuator apparatus.

6. The pipe system according to claim 1, wherein the stack is made of ceramic and has a plurality of parallel flow channels, which axially extend through the stack.

7. The pipe system according to claim 1, wherein:
the at least one energy converter includes a plurality of energy converters;
the at least one acoustic wave amplifier/attenuator apparatus includes a plurality of acoustic wave amplifier/attenuator apparatuses.

8. An acoustic wave amplifier/attenuator apparatus that is arranged in a pipe arrangement, in which an acoustic wave is propagated, and that uses thermoacoustic effect to amplify or attenuate the acoustic wave, the acoustic wave amplifier/attenuator apparatus comprising:
a cold heat exchanger;
a hot heat exchanger; and
a stack that is held between the cold heat exchanger and the hot heat exchanger.

9. The acoustic wave amplifier/attenuator apparatus according to claim 8, wherein on of the stack is in a range of 1-20.

10. A manufacturing method of a pipe system, the method comprising:
installing a sound source device to a pipe arrangement;
installing at least one energy converter to the pipe arrangement, wherein each energy converter converts energy of an acoustic wave, which is propagated in the pipe arrangement, into another form of energy; and
installing at least one acoustic wave amplifier/attenuator apparatus in the pipe arrangement at a corresponding location between the sound source device and a corresponding one of the at least one energy converter, wherein each acoustic wave amplifier/attenuator apparatus uses thermoacoustic effect to amplify or attenuate the acoustic wave and includes:
a cold heat exchanger;
a hot heat exchanger; and
a stack that is held between the cold heat exchanger and the hot heat exchanger.
